(19) United States
(12) Patent Application Publication
(10) Pub. No.: US 2003/0166614 A1
(43) Pub. Date: Sep. 4, 2003

(54) Method for reducing cholesterol and triglycerides

(76) Inventor: Stanley F. Harrison JR., Alexandria, VA (US)

Correspondence Address: Donald C. Casey, Suite 100, 311 North Washington Street, Alexandria, VA, 22314, US

(21) Appl. No.: 10/084,952

(22) Filed: Mar. 1, 2002

(51) Int. Cl. A61K 35/60

(52) U.S. Cl. 514/78; 424/523

(57) ABSTRACT

A method of treating excessive blood lipid levels in humans is described. The treatment includes a daily dosage of food supplements available over-the-counter without a prescription. The supplements are fish oil concentrate, niacin (flush free), and lecithin. In the preferred embodiment two 500 mg tablets of niacin, 2 1200 mg soft gels of lecithin, and two 1250 mg soft gels of fish oil concentrate are administered orally once to twice per day.

### FIELD OF THE INVENTION

This invention relates to a method for treating humans with high levels of cholesterol and/or triglycerides using a combination of over-the-counter dietary supplements.

### DESCRIPTION OF THE PRIOR ART

As is well known, increased levels of cholesterol, and in particular low density lipoproteins (LDL) is associated with circulation problems which often lead to heart attack and stroke. The increased level of cholesterol often is also accompanied by an increased level of triglycerides in the circulatory system. Increased plasma lipid levels have been associated with the build up of plaque within blood vessels, and there are a variety of treatments known to reduce these levels. Some treatments are more effective than others, and most are associated with undesirable side effects in many patients.

The total cholesterol level includes both the beneficial high density lipoproteins (HDL) and the troublesome low density lipoproteins, (LDL). Measurement of the total cholesterol level then can be misleading because if the LDL concentration is low and the HDL is also low this can be an undesirable situation. It is necessary to have the HDL concentration above a preset value, and the LDL concentration below a preset value, and both must be measured to provide an accurate picture.

It has been proposed to control cholesterol level merely with a strict dietary regimen. In most instances, however, diet alone cannot achieve the desired result. It is necessary to use an additional drug in order to achieve the healthy cholesterol and triglyceride levels. See The Niacin Solution, William B. Parsons, Jr. (Lilac Press 1998).

It has been proposed especially with patients who have suffered a heart attack to take aspirin on a daily basis. Most studies indicate that aspirin has been beneficial and its use encouraged. In many patients, however, aspirin alone is insufficient.

In general there are three groups of drugs on the market used for controlling cholesterol. They are bile secreted resins, fibrates, and statins.

The resins act against bile acids in the intestine preventing them from being reabsorbed and recycled into cholesterol. While these drugs are capable reducing LDL cholesterol, they do not raise HDL cholesterol or lower triglycerides. The drugs further have uncomfortable side effects and can interfere with the absorption of various other drugs such as anticoagulants, diuretics, and the like. While fibrates are capable of raising triglycerides and raising HDL cholesterol they do not reduce LDL cholesterol levels. Furthermore there have been studies and in particular a double blind study in which the results were less than clinically significant.

Finally, the drugs most commonly used are statins which act to alter liver functions and thereby the production of cholesterol. These drugs must be administered under a doctors care and have been associated with myopathy especially is the patient is taking another drug such as an antidepressant. Statins are metabolized by liven enzymes, and if an additional drug is taken such as an antidepressant which inhibits the liver enzymes the result can be similar to an overdose of the statin drug.

Concerning diet, it is known that a high diet of polyunsaturated fatty acids is undesirable because it reduces HDL cholesterol, and monounsaturated fatty acids have a favorable effect by reducing LDL cholesterol. In U.S. Pat. No. 5,518,753 it was proposed to provide an edible fatty acid triglyceride mixture which included fatty acids rich in linoleic acids and alpha-linolenic acids from various vegetable oils and fish oils.

In U.S. Pat. No. 5,886,037 a food composition was described using fish oil as an ingredient providing the beneficial fatty acids docosahexaenoic acid (DHA) and eicosapeiitaenoic acid (EPA) present in about 5-8% of the total composition. This patent describes then a food product and also requires a number of medium chain fatty acids and polyunsaturated fatty acids as ingredients.

Accordingly, there remains a need for a food supplement without undue side effects which can be purchased over-the-counter and is effective to reduce LDL cholesterol and triglycerides and increase HDL cholesterol.

### SUMMARY OF THE INVENTION

It has been discovered that a combination of fish oil concentrate, niacin, and lecithin taken in combination in pill form once a day will achieve the desired beneficial results with cholesterol and triglyceride reduction.

Accordingly it is an object of this invention to provide a dietary supplement which can be purchased over-the-counter without a prescription which will be effective in reducing undesirable levels of triglycerides without undue side effects.

It is another object of this invention to provide a combination of individually administered lecithin, niacin, and fish oil concentrate wherein the niacin is administered in pill form and the fish oil concentrate and lecithin in soft gel form whereby the patient can purchase these supplements and achieve a beneficial result without using prescription medication.

It is a further object of this invention to provide a combination of well known dietary supplements which together function to reduce LDL cholesterol, increase HDL cholesterol, and reduce triglycerides when administered on a daily basis within about 4-6 weeks.

These and other objects will be readily apparent with reference to the following description.

### DETAILED DESCRIPTION OF THE INVENTION

The purpose of this invention is to provide the following goals of treatment:

The total cholesterol level should be below 200 with the LDL cholesterol target below 100 for anyone with a previous cardiovascular event and below 130 for everyone else. The HDL cholesterol should be above 45 or between 40 and 45. Triglycerides should be below about 175. These values are expressed in milligrams per deciliter (mg/dL).

This invention involves the discovery that the daily oral dosage of a combination of fish oil concentrate, niacin and lecithin will result in reaching the above identified goals as well as reducing triglycerides within about 6 weeks.

Fish oil concentrate is available from a number of sources commercially in the form of soft gel dosages. Fish oil concentrate is a good source of omega-3 polyunsaturated fatty acids EPA and DHA. These fatty acids have been identified with beneficial results relative to lowering lipid levels in blood plasma.

One source of fish oil concentrate is Dale Alexander's MaxEPA or Super MaxEPA marketed by Twinlabs as a cholesterol free product containing EPA and DHA. In the MaxEPA product each pair of soft gels provides 600 mg of EPA and 240 mg of DHA in a 2500 mg dosage. In the preferred embodiment of this invention Super MaxEPA provides 450 mg EPA and 300 mg DHA in each soft gel.

The niacin ingredient preferably is a flush free product and in a preferred embodiment it is marketed in 500 mg tablets by KAL Dietary Supplements. The product is marketed as Inositol Hexanicotinate.

One of the problems with niacin when it is ingested in its pure nicotinic acid form is that the patient will experience a skin flushing due to marked dilation of the capillaries. Even doses as small as 50 mg can produced such a severe flush. The flush is confined to the head and neck but it may involve the upper half of the body or occasionally the whole body surface. The skin feels hot in the effected area and may also tingle or itch. The flash lasts perhaps 15 to 20 minutes or longer, and many patients will not tolerate this occurrence. In order to counter act this flash the niacin is chemically combined to provide a more slow dissolving product. In the preferred embodiment of this invention the niacin is present as Inositol Hexanicotinate.

If plain niacin is used, the dosage should be about twice that of the chemically combined product. Plain niacin, however, has been associated with nausea in addition to the flush and may alter liver function. Therefore the preferred embodiment of this invention is the chemically combined form.

Finally, the third ingredient is lecithin. Lecithin is a well known antioxidant and preservative, and is available as a dietary supplement in soft gels. The preferred embodiment is 1200 mg units also supplied by KAL Dietary Supplements. This lecithin product is produced from soybeans. This particular product has an average of 61% phosphatides and contains a number of polyunsaturated fatty acids.

The preferred daily dosage is two MaxEPA soft gels, two 500 mg flash free niacin tablets, and two 1200 mg lecithin soft gels.

The following examples are illustrative of this invention but are not intended to limit the scope thereof. In each instance the combination of medication was as stated above, fish oil concentrate, flush free niacin, and lecithin in the quantities described.

In the case of the first individual, the triglyceride measurement was 385, LDL cholesterol was 160 and HDL cholesterol was 43. After 6-7 weeks of taking the medication according to this invention, the triglyceride count dropped to 265, HDL cholesterol increased to 55, and the LDL cholesterol dropped to 156.

In the case of a second individual, the triglyceride measurement was 213, with total cholesterol at 164. After 6-7 weeks of the medication according to this invention the triglyceride count dropped to 134 and total cholesterol dropped to 130 with HDL cholesterol measured at 40. Subsequently, one year later, the triglyceride count was 137 with total cholesterol at 116 and HDL, cholesterol at 29. Subsequently, the measurements were maintained at triglycerides of 182, HDL cholesterol at 45, and LDL cholesterol at 77.

In the case of a third individual the initial triglyceride measurement was 404 with an HDL cholesterol measurement of 35. The triglyceride measurement was too high to permit calculation of the LDL cholesterol level. After the medication according to this invention the triglyceride level dropped to 250 with an HDL cholesterol measurement of 45 and an LDL cholesterol measurement of 75. The total cholesterol level dropped from 255 to 170. In each instance, no side effects were noted.

In summary, according to this invention, a combination of over-the-counter food supplements has been found to be effective in reducing of blood lipid levels and specifically, to reduce LDL cholesterol, increase HDL cholesterol and reduce triglycerides. The overall result then is a combination of food additives which protect against circulatory conditions leading to heart attack and stroke.

The food additives of this invention are niacin, fish oil concentrate, and lecithin. The niacin in the preferred embodiment is flush free chemically combined compound taken in the form of 500 mg tablets with two tablets being preferred per day. In the case of lecithin, soft gels are preferred, and two 1200 mg soft gels are the preferred dosage. Each 1200 mg soft gel typically supplied 192 mg phosphatidyl choline, 168 mg phosphatidyl ethanolamines and 108 mg of phosphatidyl inositol.

The third ingredient, fish oil concentrate, typically is also in soft gels of 1250 mg, and two per day are preferred. In each two soft gels there are omega 3 polyunsaturates in the form of 450 mg EPA and 200 mg DHA (eicosapentaenoic acid and docosahexaenoic acid, respectively).

In the case of each of these three food supplements, the dosage level is dictated by the commercially available dosages and the above is intended to merely illustrate a preferred embodiment rather than limit the invention to the dosage level set forth above.

It will be readily seen by one of ordinary skill in the art that the present invention fulfills all of the objects set forth above. After reading the foregoing specification, one of ordinary skill will be able to effect various changes, substitutions or equivalents and various other aspects of the invention as broadly disclosed herein. It is therefore intended that the protection granted hereon be limited only by the definition contained in the appended claims and equivalents thereof.

1. Method for treating excessive blood lipid levels in humans comprising:
daily administering orally a therapeutically effective amount of fish oil concentrate, niacin, and lecithin, in combination.

2. The method of claim 1 wherein lecithin is administered in two 1200 mg tablets.

3. The method of claim 1 wherein niacin is administered in two 500 mg soft gels.

4. The method of claim 3 wherein the niacin is present in the form of inositol hexanicotinate.

5. The method of claim 1 wherein the fish oil concentrate is present in the form of two 1250 mg soft gels.

6. The method of claim 5 wherein the two soft gels contain about 450 mg of eicosapentaenoic acid and about 300 mg of docosahexaenoic acid.

7. The method of claim 1 wherein said daily administration extends at least six to seven weeks.
